(19) United States
(12) Patent Application Publication
(10) Pub. No.: US 2015/0233051 A1
(43) Pub. Date: Aug. 20, 2015

(54) PSEUDOMONAS FLUORESCENS CGMCC#5974 AND ITS APPLICATION THEREOF

(71) Applicant: BEIJING TIAN'AN BIOSCIENCE & TECHNOLOGY CO., LTD., Beijing (CN)

(72) Inventor: Ping Jia, Inner Mongolia (CN)

(73) Assignee: BEIJING TIAN'AN BIOSCIENCE & TECHNOLOGY CO., LTD., Beijing (CN)

(21) Appl. No.: 14/428,967

(22) PCT Filed: Sep. 16, 2013
(86) PCT No.: PCT/CN2013/001086

§ 371 Date: Mar. 17, 2015

(30) Foreign Application Priority Data
Sep. 17, 2012 (CN) 201210342164.3
Sep. 17, 2012 (CN) 201210342544.7
Sep. 17, 2012 (CN) 201210342623.8

(51) Int. Cl. D06M 16/00; D21C 5/00; D21D 5/02; C12N 1/20; D21C 1/02

(52) U.S. Cl. CPC D06M 16/003; C12N 1/20; D21C 1/02; D21D 5/02; D21C 5/005; D06M 2101/06

(57) ABSTRACT

Provided are a complex microbial flora, an application thereof in preparing a textile fabric, a cellulose for use as an additive, and a biological bacterial solution pulp, and a method for using the complex microbial flora. The complex microbial flora comprises Bacillus sp. of deposit number CGMCC No. 5971, Rheinheimera tangshanensis of deposit number CGMCC No. 5972, Acinetobacter lwoffii of deposit number CGMCC No. 5973, Pseudomonas fluorescens of deposit number CGMCC No. 5974, and Wickerhamomyces anomalus of deposit number CGMCC No. 5975. The method provided comprises: formulation of a bacterial solution, processing of raw materials, and preparation or pulping of the fiber. In one embodiment, the invention generally includes at least one Pseudomonas fluorescens of deposit number CGMCC No. 5974, either alone or in the presence of other microorganisms as a complex microbial flora, and their applications thereof.

### BACKGROUND OF THE INVENTION

1. Field of the Invention

This invention generally relates to synthesis of a single and/or a complex microbial flora and application methods thereof in preparing textile fiber, additive cellulose, and a biological bacterial solution pulp.

2. Description of the Related Art

Recently, with the depletion of fossil fuel resources, the demand for hemp fiber synthesized by means with ecological and environmental friendly and many other fine features have attracted many consumers. Worldwide demand of natural fibers from natural raw materials has increased at an 8% rate per year. The most important features of the raw material of natural hemp fiber include its high fiber content, fine and long fibers (which helps the hemp fiber to interlace well), high strength; its fiber cell has small luminal, thick cell wall, and a high ratio of cell wall to luminal; it's opaque as hemp fiber has long and fine cell, etc. However, the drawback is that hemp fiber is not easy to fibrillation, which lowered the air permeability of fabric made from hemp fiber.

Prior methods of preparing fiber from hemp are mostly chemical processes. Waste and contaminants generated by chemical processes are dumped to the soil, thereby destroying the land and contaminating the air. In addition, such processes require very high power, electricity and water consumption. According to the current understanding of China's pulping industry, chemical pulp has been widely used in leading production plants, which generates contaminated waste solution, thereby drastically devastating the land and polluting the air. Chemical pulping processes require the use of large amount of sodium hydroxide (strong base) as additives and the use of harmful chemical elements and waste solutions during bleaching processes. All of the generated large amounts of waste and chemicals cannot be fully recycled or reused. Moreover, chemical pulping processes require very high power, electricity and water consumption. Most chemical pulping plants consume large amount of electricity and does not meet national energy conservation policy. Substances cannot achieve effective recycling. Chemicals cannot be separated from the waste and can only be dumped into soil to contaminate the environment. Also, most organic matters are mixed with chemicals, such that the organic matters cannot be reused, causing heavy losses. Thus, it's necessary to develop a bio-fiber technology system to solve the fundamental problems of environmental pollution in order to conserve energy, reduce waste generation, reduce water consumption, and to reduce production costs and improve the percentage of raw material usage.

Therefore, there is a need to develop and synthesize a novel biological bacterial pulping system to process raw materials and generate textile fiber, additive cellulose.

### SUMMARY OF THE INVENTION

This invention generally relates to biological bacterial species and systems to process raw materials and generate textile fiber, additive cellulose, and a biological pulping solution for making papers. More specifically, the invention relates to methods and biological systems of preparing a biological pulping solution to make papers. In one embodiment, the invention generally includes at least one Pseudomonas fluorescens of deposit number CGMCC No. 5974, either alone or in the presence of other microorganisms as a complex microbial flora, and their applications thereof.

In another embodiment, provided are a complex microbial flora, an application thereof in preparing a textile fabric, a cellulose for use as an additive, and a biological bacterial solution pulp, and a method for using the complex microbial flora. The complex microbial flora comprises Bacillus sp. of deposit number CGMCC No. 5971, Rheinheimera tangshanensis of deposit number CGMCC No. 5972, Acinetobacter lwoffii of deposit number CGMCC No. 5973, Pseudomonas fluorescens of deposit number CGMCC No. 5974, and Wickerhamomyces anomalus of deposit number CGMCC No. 5975. The method provided comprises: formulation of a bacterial solution, processing of raw materials, and preparation or pulping of the fiber. The method does not pollute the environment, and the wastewater is transformed directly into an organic fertilizer, thus achieving zero emission and zero pollution. A biological treatment process plays a protective role for the fiber, and, compared with a conventional chemical method, reduces production costs, increases economic benefits, and is energy-saving and environmentally friendly.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates an exemplary flowchart of a method of making textile fiber according to one embodiment of the invention.

FIG. 2 illustrates one embodiment of a flow chart of an exemplary method of producing additive cellulose.

FIG. 3 illustrates one embodiment of a flow chart of another exemplary method of producing additive cellulose.

FIG. 4 illustrates one embodiment of a flowchart of an exemplary method of producing a microbial biological pulping solution system.

FIG. 5 illustrates one embodiment of a flowchart of another exemplary method of producing a microbial biological pulping solution system.

### DETAILED DESCRIPTION

The present invention overcomes the drawbacks in the prior chemical processes of fiber production and generally includes novel biological pulping systems and methods of making fibers, celluloses, and paper-generating biological pulping solution systems using one or more microbial/bacterial species. The present invention generally provides at least one Bacillus. Sp. under CGMCC Deposit No. 5971, Rheinheimera tangshanensis under CGMCC Deposit No. 5972, Acinetobacter lwoffii strain under CGMCC Deposit No. 5973, Pseudomonas fluorescens under CGMCC Deposit No. 5974, and Wickerhamonyces anomalus under CGMCC Deposit No. 5975, either alone or as a complex microbial flora in the presence of other bacteria, a biological pulping solution system in the presence of one or more bacteria species, and their applications thereof.

One embodiment of the invention provides one or more bacteria species within a biological pulping solution system, including at least one Bacillus. Sp. under CGMCC Deposit No. 5971, Rheinheimera tangshanensis under CGMCC Deposit No. 5972, Acinetobacter lwoffii strain under CGMCC Deposit No. 5973, Pseudomonas fluorescens under CGMCC Deposit No. 5974, and Wickerhamonyces anomalus under CGMCC Deposit No. 5975 and their combinations thereof, either alone or in the presence of one or more other bacteria.

For example, the biological pulping solution system may include a complex microbial flora of Bacillus. Sp. CGMCC Deposit No. 5971 and Rheinheimera tangshanensis CGMCC Deposit No. 5972; a complex microbial flora of Bacillus. Sp. CGMCC Deposit No. 5971 and Acinetobacter lwoffii strain CGMCC Deposit No. 5973; a complex microbial flora of Bacillus. Sp. CGMCC Deposit No. 5971 and Pseudomonas fluorescens CGMCC Deposit No. 5974; a complex microbial flora of Bacillus. Sp. CGMCC Deposit No. 5971 and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, and Acinetobacter lwoffii strain CGMCC Deposit No. 5973; a complex microbial flora of Bacillus. Sp. CGMCC Deposit No. 5971, Rheinheimera tangshanensis CGMCC Deposit No. 5972, and Pseudomonas fluorescens CGMCC Deposit No. 5974; a complex microbial flora of Bacillus. Sp. CGMCC Deposit No. 5971, Rheinheimera tangshanensis CGMCC Deposit No. 5972, and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, and Pseudomonas fluorescens CGMCC Deposit No. 5974; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, and Wickerhamonyces anomalus CGMCC Deposit No. 5975; a complex microbial flora of Bacillus. Sp. CGMCC Deposit No. 5971, Acinetobacter lwoffii strain CGMCC Deposit No. 5973, Pseudomonas fluorescens CGMCC Deposit No. 5974, and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof.

As another example, the biological pulping solution system may include a complex microbial flora of Rheinheimera tangshanensis CGMCC Deposit No. 5972, Acinetobacter lwoffii strain CGMCC Deposit No. 5973, and Pseudomonas fluorescens CGMCC Deposit No. 5974; a complex microbial flora of Rheinheimera tangshanensis CGMCC Deposit No. 5972, Acinetobacter lwoffii strain CGMCC Deposit No. 5973, and Wickerhamonyces anomalus CGMCC Deposit No. 5975; a complex microbial flora of Rheinheimera tangshanensis CGMCC Deposit No. 5972, Pseudomonas fluorescens CGMCC Deposit No. 5974, and Wickerhamonyces anomalus CGMCC Deposit No. 5975; a complex microbial flora of Acinetobacter lwoffii strain CGMCC Deposit No. 5973, Pseudomonas fluorescens CGMCC Deposit No. 5974, and Wickerhamonyces anomalus CGMCC Deposit No. 5975; a complex microbial flora of Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof.

Another embodiment of the invention provides the preparation of one or more bacteria species as a bacterial culture and the use of the bacterial cultures to prepare textile fibers, celluloses (to be used as an additive), and/or a biological pulping solution system (for making paper). In one aspect, a method of preparing a bacterial culture (e.g., a biological bacterial culture solution) to be used to make textile fibers, cellulose, and a biological pulping solution system, among others, includes growing one or more microbial or bacterial species in a malt-agar and culturing for a period of time (e.g., at 25° C. for three days). The resulting bacterial cell colonies may be in a spherical, oval, or sausage shape at a size of (4.8-14.4) μm×(3.6-7.2) μm, with possible formation of precipitates. When the one or more bacteria species are placed on malt-agar plate and cultured at 25° C. for one month, the bacterial colonies look cheese-like with white, smooth, non-reflective surface and root-like edges. When the one or more bacteria species are cultured in Dalmau cornmeal agar plate culture, false hyphae are grown on the surface.

In another aspect, a method for preparing a textile fiber includes the following one or more steps:

The first step of (1) Bacteria Configurations in a process of making textile fibers: preparing one or more bacteria species in a bacterial culture solution. Bacillus. Sp. CGMCC Deposit No. 5971, Rheinheimera tangshanensis under CGMCC Deposit No. 5972, Acinetobacter lwoffii strain under CGMCC Deposit No. 5973, Pseudomonas fluorescens under CGMCC Deposit No. 5974, and Wickerhamonyces anomalus under CGMCC Deposit No. 5975, can be present alone or in combination with other bacteria as a complex microbial flora. For example, a bacterial culture solution is prepared according to a desired weight ratio of Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof.

As an example, a complex bacterial flora is prepared in a weight ratio of: Bacillus sp.:Rheinheimera tangshanensis:Acinetobacter lwoffii at a weight ratio of 2-3:1-2:1-2; Bacillus sp.:Rheinheimera tangshanensis:Pseudomonas fluorescens:at a weight ratio of 2-3:1-2:1-2; Bacillus sp.:Rheinheimera tangshanensis:Wickerhamomyces anomalus at a weight ratio of 2-3:1-2:2-3; Bacillus sp.:Acinetobacter lwoffii:Pseudomonas fluorescens at a weight ratio of 2-3:1-2:1-2; Bacillus sp.:Acinetobacter lwoffii:Wickerhamomyces anomalus at a weight ratio of 2-3:1-2:2-3; Bacillus sp.:Pseudomonas fluorescens:Wickerhamomyces anomalus at a weight ratio of 2-3:1-2:2-3; Bacillus:Rheinheimera tangshanensis:Wickerhamomyces anomalus at a weight ratio of 2-3:1-2:2-3; Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Wickerhamomyces anomalus at a weight ratio of 2-3:1-2:1-2:2-3; Bacillus sp.:Rheinheimera tangshanensis:Pseudomonas fluorescens:Wickerhamomyces anomalus at a weight ratio of 2-3:1-2:1-2:2-3; Bacillus sp.:Acinetobacter lwoffii:Pseudomonas fluorescens:Wickerhamomyces anomalus at a weight ratio of 2-3:1-2:1-2:2-3; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii: Pseudomonas fluorescens at a weight ratio of 1-2:1-2:1-2; Rheinheimera tangshanensis:Acinetobacter lwoffii:Wickerhamomyces anomalus at a weight ratio of 1-2:1-2:2-3; Rheinheimera tangshanensis:Pseudomonas fluorescens:Wickerhamomyces anomalus at a weight ratio of 1-2:1-2:2-3; Acinetobacter lwoffii:Pseudomonas fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3.

The second step of (2) Preparation of raw materials: preparing and cutting hemp raw materials into fragments and sections, and soaking the raw materials inside a swelling pool to swell the fragments of hemp raw materials. The raw materials may include hemp flax, sesame, jute, sisal, combinations thereof, among others. The swelled raw materials are then taken out of the swelling pool for further processing.

The third step of (3) Fiber Production: Fiber Production may include the following step of (a) Biodegradation in a biological system: by removing and draining the above biodegraded materials and soaking the prepared raw materials into the prepared bacterial culture solution. (b) Steam sterilization: by removing and draining the above biodegraded materials from the bacteria culture solution, and sterilizing the biodegraded materials (e.g., by passing the biodegraded materials through steam sterilization). (c) Obtaining Fiber: coarsely grinding the sterilized material for a period of time to obtain fiber bundles, then finely grinding the fiber bundles to disperse the fiber bundles into individual single fibers. Next, screening and filtering the fiber bundles and re-grinding them repeatedly for several times until all of them are grinded into individual single fibers. (d) Drying and carding: soaking the above-obtained single fibers in warm water, drying and combing the single fibers to use them for making textile fibers.

Additional embodiment of the invention provides the preparation of celluloses to be used as additives and includes the following steps: (1) Bacteria Configurations in a process of making cellulose additives: preparing one or more bacteria species in a bacterial culture solution. Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975 can be present alone or in combination with other bacteria as a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof. A bacterial culture solution is prepared according to a desired weight ratio, which can be any of the weight ratios as described above or any other desired ratios.

The second step of (2) Preparation of raw materials: debarking woody raw material and cutting them into pieces, sections, and fragments; and/or cutting herbal raw materials into fragments and sections. Then, ingredients of the chopped materials are soaked inside a swelling pool to swell the chopped raw materials. The raw materials may include any woody plant tissues, chipped wood or tree tissues, processed wood products, herbal plant tissues, chopped fiber-rich plant tissues, or any combinations thereof. The swelled raw materials are then taken out of the swelling pool for further processing.

The third step of (3) Fiber Production: Fiber Production may include the following step of (a) Easing the raw material: rolling and/or squeezing the swelled raw materials. (b) Biodegradation in a biological system: by placing the eased raw materials to be soaked in a prepared bacterial culture solution. (c) Steam sterilization: by removing and draining the above biodegraded materials from the bacteria culture solution, and sterilizing the biodegraded materials (e.g., by passing the biodegraded materials through steam sterilization). (d) Obtaining Fiber: coarsely grinding the sterilized material for a period of time to obtain fiber bundles, then finely grinding the fiber bundles to disperse the fiber bundles into individual single fibers. Next, screening and filtering the fiber bundles and re-grinding them repeatedly for several times until all of them are grinded into individual single fibers. (e) Sterilization: soaking the above-obtained single fibers in warm water, and then drying and sterilization. (f) Grinding: grinding the sterilized fiber into celluloses, which can be used as additives.

This invention also provides a method for biologically preparing a pulp solution system (e.g., a paper-making pupping solution) using one or more bacterial species. The method includes the following steps: (1) Bacteria Configurations within the biological pulping solution system: preparing the above one or more bacteria species into a bacterial culture according to the desired weight ratio and proportion. The bacteria in a bacteria culture solution can be present alone or in combination with other bacteria as a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof. A bacterial culture solution is prepared according to a desired weight ratio.

The second step of (2) Preparation of raw materials: debarking woody raw material and cutting them into pieces, sections, and fragments; and/or cutting herbal raw materials into fragments and sections. Then, ingredients of the chopped materials are soaked inside a swelling pool to swell the chopped raw materials. The raw materials may include any woody plant tissues, chipped wood or tree tissues, processed wood products, herbal plant tissues, chopped fiber-rich plant tissues, or any combinations thereof. The swelled raw materials are then taken out of the swelling pool for further processing.

The third step of (3) Pulp Production: Pulp Production may include the following step of (a) Easing the raw material: rolling and/or squeezing the swelled raw materials. (b) Biodegradation in a biological pulping system: by placing the eased raw materials to be soaked in a prepared bacterial culture solution. (c) Steam sterilization: by removing and draining the above biodegraded materials from the bacteria culture solution, and sterilizing the biodegraded materials (e.g., by passing the biodegraded materials through steam sterilization). (d) Coarse Pulping: coarsely grinding the sterilized material for a period of time to obtain fiber bundles. (e) Fine Pulping: finely grinding the fiber bundles to disperse the fiber bundles into individual single fibers. (f) Pulp Screening: screening and filtering the fiber bundles and re-grinding them several times until all of them are grinded into individual single fibers. (g) Pulp Washing: Soaking the obtained pulp in warm water for a period of time and use the obtained pulp to make paper (e.g., paperboard, etc).

The method for biologically preparing a paper-making pulp solution system as described above may contain, for example, a bacteria culture at a density of 60,000,000/ml at step (1), a raw material swelling time of about 10 to 12 hours at step (2), and a biodegradation temperature to be maintained at about 35-40° C. for 32-36 hours at step (3). After the Easing step, the mass ratio of raw materials and the bacteria solution is 1:6-9. Steam sterilization is performed at atmospheric pressure for 10-30 minutes using water vapor sterilization.

Furthermore, at step (2), after the raw materials are taken out of the swelling pool, the solution within the swelling pool can be further processed to be flocculated and get rid of sediments to obtain upper layer clear supernatant solution. The clear solution can be recycled and used again. The sedimentary materials can be abated into a biogas pool to generate biogas.

The invention provides many advantages and characteristics, including: (1) The methods described herein do not pollute the environment. The generated waste can be directly transformed into organic fertilizers, thereby obtaining zero emissions, zero pollution to the environment. (2) The fibers generated by the biological pulping method are protected. As compared to the conventional chemical processes, the methods described herein can obtain almost all cellulose and hemicellulose from the raw materials, thereby obtaining high fiber production yield. (3) The biological degradation process is conducted under atmospheric pressure to save energy, use a low carbon technology, and reduce emission. (4) The costs of a biological process is lower, thus high economic effect.

The by-products of the processes of the present invention can be delivered to a precipitation tank to be flocculated and precipitated. Then, the supernatant clear solution is recycled and reused as a pre-soak water solution. The flocculates, sediments and precipitates are rich in a variety of organic matters and many other nitrogen, phosphorus, or potassium-containing phytonutrients. The flocculates, sediments and precipitates can be mixed with the used, old bacteria culture solution (namely the viscous bacteria culture solution that has been repeatedly used to degrade raw materials, which also contain nitrogen, phosphorus, potassium, iron and trace elements). The mixture, after acidification, can be discharged into a bio-fermentation tank to produce biogas. The generated biogas residues, biogas liquids, grinded ashes, mixed particles, and other wastes can also be mixed to generate organic fertilizers or be abated, resulting in zero pollutant emission to the environment.

The methods described herein further improve cellulose production and pulping production, reduces the reaction time, and increases the purity of the obtained fiber and their yields, as compared to chemical processes, so that the methods described herein can promote large-scale application in the actual production. The methods described herein use a bacteria culture solution to degrade plant tissues and obtain plant fibers in a short time. The methods described herein can also be used to biologically degrade plant lignin within a short time to produce pulp and make paper. The by-products of the process can be converted into biogas, which can be used to heat a boiler (which may use coal and biogas to heat), thereby saving coal consumption. Lastly, biogas residues and wastes can be made into organic fertilizers, thereby forming a new economic recycle model for “organic material transformation” (where substances are transformed into organic matters) and no discharging of any waste materials. As a result, the invention fundamentally solves pollution problems as seen in prior art chemical fiber preparation process, conserves energy, reduces emission, saves water, reduces production costs, and improves material utilization efficiency.

### Example 1

### The Preparation of a Bacteria Culture Solution

One or more bacteria species as described herein have been deposited on Apr. 6, 2012 into the China General Microbiological Culture Collection Center (CGMCC, located at No. 3, Division #1, Beichen West Road, Chaoyang District, Beijing, China). The deposit numbers are Bacillus sp. Deposit No. 5971, Rheinheimera tangshanensis Deposit No. 5972, Acinetobacter lwoffii CGMCC Deposit No. 5973, Pseudomonas fluorescens CGMCC Deposit No. 5974, and Wickerhamomyces anomalus Deposit No. 5975. The bacteria used herein in a bacteria culture solution can include at least one Bacillus. Sp. under CGMCC Deposit No. 5971, Rheinheimera tangshanensis under CGMCC Deposit No. 5972, Acinetobacter lwoffii strain under CGMCC Deposit No. 5973, Pseudomonas fluorescens under CGMCC Deposit No. 5974, and Wickerhamonyces anomalus under CGMCC Deposit No. 5975 and their combinations thereof, either alone or in the presence of one or more other bacteria.

For example, the bacteria culture solution may include a complex microbial flora of Bacillus. Sp. CGMCC Deposit No. 5971 and Rheinheimera tangshanensis CGMCC Deposit No. 5972; a complex microbial flora of Bacillus. Sp. CGMCC Deposit No. 5971 and Acinetobacter lwoffii strain CGMCC Deposit No. 5973; a complex microbial flora of Bacillus. Sp. CGMCC Deposit No. 5971 and Pseudomonas fluorescens CGMCC Deposit No. 5974; a complex microbial flora of Bacillus. Sp. CGMCC Deposit No. 5971 and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, and Acinetobacter lwoffii strain CGMCC Deposit No. 5973; a complex microbial flora of Bacillus. Sp. CGMCC Deposit No. 5971, Rheinheimera tangshanensis CGMCC Deposit No. 5972, and Pseudomonas fluorescens CGMCC Deposit No. 5974; a complex microbial flora of Bacillus. Sp. CGMCC Deposit No. 5971, Rheinheimera tangshanensis CGMCC Deposit No. 5972, and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, and Pseudomonas fluorescens CGMCC Deposit No. 5974; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, and Wickerhamonyces anomalus CGMCC Deposit No. 5975; a complex microbial flora of Bacillus. Sp. CGMCC Deposit No. 5971, Acinetobacter lwoffii strain CGMCC Deposit No. 5973, Pseudomonas fluorescens CGMCC Deposit No. 5974, and Wickerhamonyces anomalus CGMCC Deposit No. 5975; a complex microbial flora of Bacillus. Sp. CGMCC Deposit No. 5971, Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof.

As another example, the bacteria culture solution may include a complex microbial flora of Rheinheimera tangshanensis CGMCC Deposit No. 5972, Acinetobacter lwoffii strain CGMCC Deposit No. 5973, and Pseudomonas fluorescens CGMCC Deposit No. 5974; a complex microbial flora of Rheinheimera tangshanensis CGMCC Deposit No. 5972, Acinetobacter lwoffii strain CGMCC Deposit No. 5973, and Wickerhamonyces anomalus CGMCC Deposit No. 5975; a complex microbial flora of Rheinheimera tangshanensis CGMCC Deposit No. 5972, Pseudomonas fluorescens CGMCC Deposit No. 5974, and Wickerhamonyces anomalus CGMCC Deposit No. 5975; a complex microbial flora of Acinetobacter lwoffii strain CGMCC Deposit No. 5973, Pseudomonas fluorescens CGMCC Deposit No. 5974, and Wickerhamonyces anomalus CGMCC Deposit No. 5975; a complex microbial flora of Rheinheimera tangshanensis CGMCC Deposit No. 5972, Acinetobacter lwoffii strain CGMCC Deposit No. 5973, Pseudomonas fluorescens CGMCC Deposit No. 5974, and Wickerhamonyces anomalus CGMCC Deposit No. 5975; and any combinations thereof.

The bacteria culture solution can be cultured into a density of about 60,000,000/ml or above to be used in the methods described herein. A complex microbial flora present in a bacterial culture solution can be prepared according to any of a desired weight ratio of the bacteria present.

For example, a complex bacterial flora can be prepared in a weight ratio of: Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii:Pseudomonas fluorescens at a weight ratio of 1-2:1-2:1-2; Bacillus:Rheinheimera tangshanensis:Acinetobacter lwoffii:Pseudomonas fluorescens:Wickerhamomyces anomalus at a weight ratio of 3:1:2:2:2; Bacillus sp.:Acinetobacter lwoffii:Pseudomonas fluorescens:Wickerhamomyces anomalus at a weight ratio of 2-3:1-2:1-2:2-3; Bacillus sp.:Acinetobacter lwoffii:Pseudomonas fluorescens at a weight ratio of 2-3:1-2:1-2; Acinetobacter lwoffii:Pseudomonas fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3. As another example, a complex bacterial flora is prepared in a weight ratio of. Bacillus sp.:Rheinheimera tangshanensis:Pseudomonas fluorescens.:at a weight ratio of 2-3:1-2:1-2; Bacillus sp.:Rheinheimera tangshanensis:Pseudomonas fluorescens:Wickerhamomyces anomalus at a weight ratio of 2-3:1-2:1-2:2-3; Rheinheimera tangshanensis:Pseudomonas fluorescens:Wickerhamomyces anomalus at a weight ratio of 1-2:1-2:2-3; and Bacillus sp.:Pseudomonas fluorescens:Wickerhamomyces anomalus at a weight ratio of 2-3:1-2:2-3.

As another example, a complex bacterial flora is prepared in a weight ratio of Bacillus sp.:Rheinheimera tangshanensis:Acinetobacter lwoffii at a weight ratio of 2-3:1-2:1-2; Bacillus sp.:Rheinheimera tangshanensis:Acinetobacter lwoffii:Wickerhamomyces anomalus at a weight ratio of 2-3:1-2:1-2:2-3; Bacillus sp.:Rheinheimera tangshanensis:Wickerhamomyces anomalus at a weight ratio of 2-3:1-2:2-3; Bacillus sp.:Acinetobacter lwoffii:Wickerhamomyces anomalus at a weight ratio of 2-3:1-2:2-3; and Rheinheimera tangshanensis:Acinetobacter lwoffii:Wickerhamomyces anomalus at a weight ratio of 1-2:1-2:2-3.

Bacillus:Rheinheimera tangshanensis:Acinetobacter lwoffii:Pseudomonas fluorescens:Wickerhamomyces anomalus at a weight ratio of 3:1:1:2:3. As another example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 3:1:2:2:2. As another example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 3:2:1:2:2.

As another example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 2:1:2:2:3. As another example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 2:2:1:2:3. Any other desired combinations and desired weight ratios can also be used.

### Example 2

### Extraction of Fibers from Flax

A method of making fibers by using flax as an example of raw materials: a bacterial culture solution is prepared according to a desired weight ratio. As an example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 3:1:1:2:3.

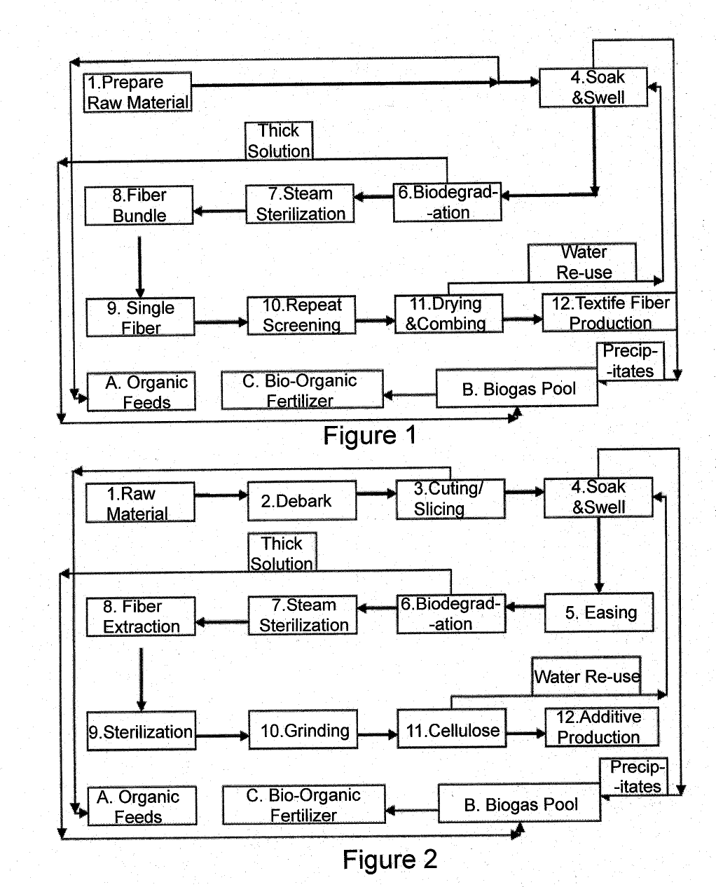

According to the embodiments of FIG. 1, the process of fiber extraction generally includes three phases: a preparation phase, a fiber-manufacturing phase and a by-product generating phase.

(A). A Preparation Phase:

from Step 1 (prepare raw material) to Step 4 (soak & swell.

The flax raw material was harvested, prepared, and then soaked & washed with cold water in a soaking, wetting, retting pool or tank so as to first wash away any dirt and other debris on the surface of the raw materials. The flax raw materials were soaked with water at natural temperature until the flax raw materials were totally retted and swelled, for example, for about 10-12 hours. After repeat soaking, the solution liquid became turbid. Then, after flocculation and sedimentation, the supernatant clear solution was recycled for re-use. The precipitates and sediments were discharged and abated into a biogas tank for fermentation and producing biogas.

(B) a Fiber-Manufacturing Phase: From Step 6 (Biodegradation) to Step 12 (Textile Fiber Production.

Step (6) Biodegradation: The treated raw materials were put into a biodegradation bin or pot to be soaked and mixed inside a bacterial culture solution (for example, the bacterial culture solution as prepared in Example 1). The mass ratio of the raw materials and the bacterial culture solution is 1:8. The temperature was maintained at about 35-40° C. for a time period of about 32-36 hours. Generally, the conditions of biological degradation reaction are adjusted according to the growth conditions of the bacterial culture used so as to increase specific bacterial degradation.

Step (7) Steam Sterilization: Biodegraded materials were removed from the degradation pot, drained, and sterilized to remove bacteria. Then, the biodegraded materials were placed inside a steam pot and steam was used to passing through them for about 10-30 minutes. The biodegraded materials were then delivered from the output of the steam pot into a fiber refiner/grinder.

Step (8) Fiber Bundle Production: Fiber bundles were obtained by coarsely grinding the sterilized materials for a time period.

Step (9) Single Fiber Production: The fiber bundles were finely grinded and dispersed into single fibers.

Step (10) Repeat Screening: The fiber bundles were repeatedly screened, filtered and re-grinded again to obtain individual single fibers.

Step (11) Drying and Combing: The above-obtained fibers were soaked in warm water, then gone through drying, combing, drafting to further obtain straight and paralleled fibers.

Step (12) Fiber Extraction & Production: The above-obtained single fibers were extracted using further manufacturing techniques to obtain textile fibers.

(C) A By-Product Generating Phase: Step A-Step C.

Step A: Organic Feeds. The remainders of prepared raw materials between step (1) and (4) are rich in nutrients and can be fermented into organic feeds for cattle and sheep.

Step B: Bio-organic fertilizer. After repeated soaking and swelling at Step 4, the biodegraded material solution became turbid and was delivered to go through flocculation and sedimentation. The resulting supernatant solution was recycled for future use. Then, the precipitates and sediments were discharged and delivered into a biogas pool to be fermented and produce biogas, which can be used to heat a coal and biogas compatible boiler and reduce coal consumption.

Step C: Bio-organic Fertilizer. After fermentation in the biogas pool, the generated biogas residues, slurries, biogas liquids are rich bio-organic fertilizers. So the liquid residue/slurry solutions can be used as fertilizers for agricultural crops and nutrient solutions for flowers. The remaining solid particles and residues can be used as base fertilizers. All such fertilizers are green fertilizers. The physical properties and measurements of the obtained fibers are shown in Table 1.

### Example 3

### Extraction of Celluloses from Ramie

The preparation process is similar to those described in Example 2, except that the ratio of a complex microbial flora bacterial culture was configured as followed. As an example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 3:1:2:2:2. During biodegradation, the weight ratio of the treated raw materials and the bacteria culture solution is 1:7. The physical properties and measurements of the obtained fibers are shown in Table 1.

### Example 4

### Extraction of Celluloses from Jute and Kenaf

The preparation process is similar to those described in Example 2, except that the ratio of a complex microbial flora bacterial culture was configured as followed. As an example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 3:2:1:2:2. During biodegradation, the weight ratio of the treated raw materials and the bacteria culture solution is 1:8.5. The physical properties and measurements of the obtained fibers are shown in Table 1.

### Example 5

### Extraction of Celluloses from Sisal Hemp

The preparation process is similar to those described in Example 2, except that the ratio of a complex microbial flora bacterial culture was configured as followed. As an example, a complex bacterial flora is prepared in a weight ratio of Bacillus:Rheinheimera tangshanensis:Acinetobacter lwoffii:Pseudomonas fluorescens:Wickerhamomyces anomalus at a weight ratio of 2:1:2:2:3. During biodegradation, the weight ratio of the treated raw materials and the bacteria culture solution is 1:9. The physical properties and measurements of the obtained fibers are shown in Table 1.

TABLE 1

| | flax | ramie | jute and kenaf | sisal hemp |
|---|---|---|---|---|
| Thickness (dtex) | 5 | 4.5 | 4.3 | 4.2 |
| Breakage length (km) | 6.33 | 6.07 | 4.18 | 3.35 |
| Breakage intensity (cN/dtex) | 11 | 9.4 | 9.5 | 9.3 |
| Breakage Extension (%) | 5 | 4.5 | 3.8 | 4 |

### Example 6

### Extraction of Celluloses from Woody Materials

Using Caragana as an example of raw materials, a process is described herein to specifically illustrate how to extract cellulose from woody materials. Other woody materials, such as poplar and willow, can also be used to extract celluloses accordingly.

When using caragana as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:1:2:2:2. When using poplar as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 2:1:2:2:3. When using willow as raw material, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:2:1:2:2.

According to the embodiments of FIG. 2, the process of fiber extraction generally includes three phases: a preparation phase, a fiber-manufacturing phase and a by-product generating phase.

(A). A Preparation Phase:

from Step 1 (prepare raw material) to Step 4 (soak & swell.

Step (1) to Step (2) Preparing raw materials and Debarking: The stem and bark of the harvested caragana raw materials were separated (e.g., using winnowing machine or other machines). The bark was separated and fed into organic feed processing plant to be processes into feeds, and the debarked stem was delivered to a cutting machine.

Step (3) Cutting/Slicing: The caragana stem raw materials were cut into segments, each at a length of about 3-4 cm, preferably in oblique cut, in order to increase the area for water penetration.

Step (4) Soaking & Washing: The harvested caragana stem materials were soaked with cold water in a retting pool or tank and washed to get rid of dirt and other debris on its surface. Then, the caragana stem materials were soaked in water at natural temperatures for a time period of about 10-12 hours, or until the stem are totally retted and swelled. After repeat soaking, the solution liquid became turbid. Then, after flocculation and sedimentation, the supernatant clear solution was recycled for re-use. The precipitates and sediments were discharged and abated into a biogas tank for fermentation and producing biogas.

The above four Steps can be performed in separate batches from time to time or step-by-step continuously.

(B) a Fiber-Manufacturing Phase: From Step 5 (Easing) to Step 12 (Additive Cellulose Production.

Step (5) Easing: The soaked and swelled stem raw materials are delivered into a threading and rolling machine or a roller to be rolled and squeezed and change into woody filament structures so that the bacteria can easily penetrate inside and degrade the raw materials.

Step (6) Biodegradation: The treated raw materials were put into a biodegradation bin or pot to be soaked and mixed inside a bacterial culture solution (for example, the bacterial culture solution as prepared in Example 1). The mass ratio of the eased raw materials and the bacterial culture solution is 1:6. The temperature was maintained at about 35-40° C. for a time period of about 28-32 hours. Generally, the conditions of biological degradation reaction are adjusted according to the growth conditions of the bacterial culture used so as to increase specific bacterial degradation.

Step (7) Steam Sterilization: Biodegraded materials were removed from the degradation pot, drained, and sterilized to remove bacteria. Then, the biodegraded materials were placed inside a steam pot and steam was used to passing through them for about 10-30 minutes. The biodegraded materials were then delivered from the output of the steam pot into a fiber refiner/grinder.

Step (8) Fiber Extraction: Fiber bundles were obtained by coarsely grinding the sterilized materials for a time period. The fiber bundles were then finely grinded and dispersed into single fibers. The fiber bundles were repeatedly screened, filtered and re-grinded again to obtain individual single fibers.

Step (9) Sterilization: The fibers were mechanically grinded after coarse and fine grinding. Most of the fibers were curved, twisted, and deformed, and were extracted by soaking in warm water to change, draft and extend any curved structures formed during mechanical fiber extraction. Then, drying and sterilization of the above-obtained fibers were soaked in warm water, then gone through drying, combing, drafting to further obtain straight and paralleled fibers.

Step (10) Textile Fiber Extraction & Production: The above-obtained single fibers were extracted using manufacturing techniques known to people skilled in the art (e.g., diluted alkali or low base solution techniques) to remove lignin, sterilize, grind and obtain textile fibers at step (11). The resulting single fibers can be used as additives (12) in food or medical, or consumer products.

(C) A By-Product Generating Phase: Step A-Step C.

Step A: Organic Feeds. The barks of the prepared Caragana raw materials at Step (3) are rich in nutrients and can be fermented into feeds for cattle and sheep.

Step B: Bio-organic fertilizer. After repeated soaking and swelling at Step (4), the biodegraded material solution became turbid and was delivered to go through flocculation and sedimentation. The resulting supernatant solution was recycled for future use. Then, the precipitates and sediments were discharged and delivered into a biogas pool to be fermented and produce biogas, which can be used to heat a coal and biogas compatible boiler and reduce coal consumption.

Step C: Bio-organic Fertilizer. After fermentation in the biogas pool, the generated biogas residues, slurries, biogas liquids are rich bio-organic fertilizers. So the liquid residue/slurry solutions can be used as fertilizers for agricultural crops and nutrient solutions for flowers. The remaining solid particles and residues can be used as base fertilizers. All such fertilizers are green fertilizers. The physical properties and measurements of the obtained celluloses are shown in Table 2.

TABLE 2

| | Caragana | poplar | willow |
|---|---|---|---|
| Polymer strength | 560 | 535 | 520 |
| Surface Capacity | 7.5 | 7.2 | 7.1 |
| (cm³/g) | | | |
| Average particle | 210 | 185 | 190 |
| size (μm) | | | |

### Example 7

### Extraction of Celluloses from Herbal Plant Raw Materials

Using wheat straw as an example of raw materials, a process is described herein to specifically illustrate how to extract cellulose from herbal plant materials. Other herbal materials, such as rice straw and reeds, can also be used to extract celluloses accordingly.

When using wheat straw as an example of raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:2:1:2:2. When using rice straw as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 2:2:2:2:2. When using reeds as raw material, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:1:1:3:2.

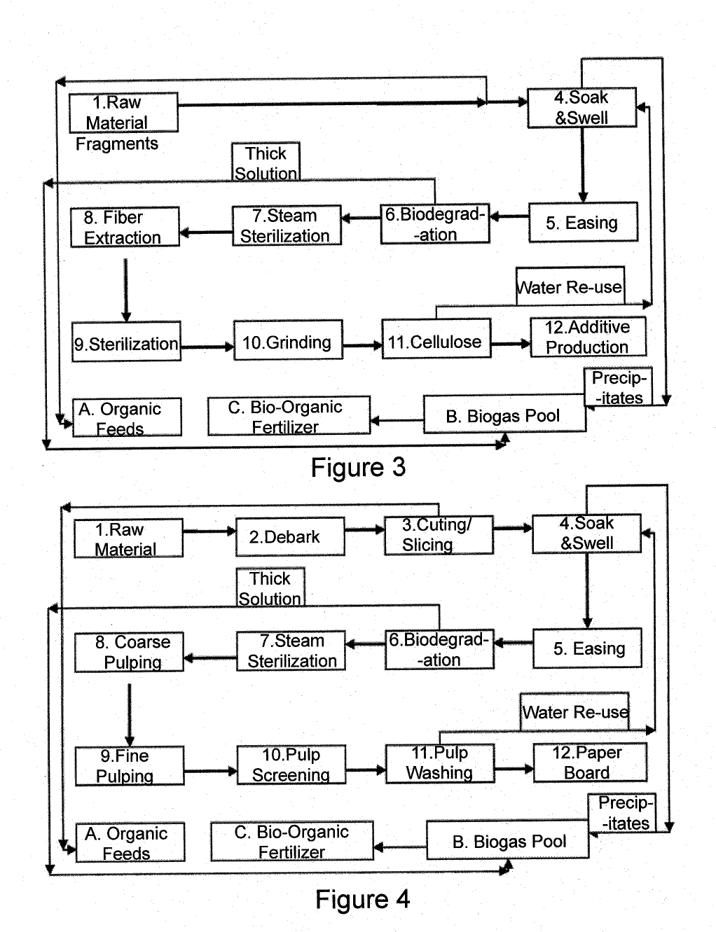

According to the embodiments of FIG. 3, the process of fiber extraction generally includes three phases: a preparation phase, a fiber-manufacturing phase and a by-product generating phase.

(A). A Preparation Phase:

from Step 1 (prepare raw material) to Step 4 (soak & swell.

Wheat straw raw materials were cut into segments, each at a length of about 4-5 cm, and then soaked & washed with cold water in a soaking, wetting, retting pool or tank so as to first wash away any dirt and other debris on the surface of the raw materials. The wheat straw raw materials were soaked with water at natural temperature until the wheat straw raw materials were totally retted and swelled, for example, for about 10-12 hours. After repeat soaking, the solution liquid became turbid. Then, after flocculation and sedimentation, the supernatant clear solution was recycled for re-use. The precipitates and sediments were discharged and abated into a biogas tank for fermentation and producing biogas.

(B) A Fiber-Manufacturing Phase:

from Step 5 (Easing) to Step 12 (Additive cellulose production; and (C) A by-product generating phase: Step A-Step C as seen in Example 2. The bacteria culture solution used are prepared the same as described in Example 1. During the biodegradation process, the weight ratio of the treated raw materials and the bacteria culture solution is 1:8. The physical properties and measurements of the obtained fibers are shown in Table 3 as below.′

TABLE 3

| | Wheat straw | Rice straw | Reeds |
|---|---|---|---|
| Polymer strength | 485 | 460 | 450 |
| Surface Capacity | 7.2 | 6.8 | 6.9 |
| (cm³/g) | | | |
| Average Particle | 180 | 160 | 168 |
| Size (μm) | | | |

### Example 8

### A Biological Pulping Process Using a Bacterial Culture and Woody Raw Materials

Using Caragana as an example of raw materials, a process is described herein to specifically illustrate how to prepare a biological pulping solution system from woody materials. Other woody materials, such as poplar and eucalyptus, etc., can also be used to in a method of preparing a biological pulping solution system accordingly.

When using caragana as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:1:2:2:2. When using poplar as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 3:2:1:2:2. When using eucalyptus as raw material, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 2:2:1:2:3.

According to the embodiments of FIG. 4, the process of making a biological pulping solution system generally includes three phases: a preparation phase, a pulp-manufacturing phase and a by-product generating phase.

(A). A Preparation Phase:

from Step 1 (prepare raw material) to Step 4 (soak & swell.

Step (1) to Step (2) Preparing raw materials and Debarking: The stem and bark/epiderm of the harvested caragana raw materials were separated (e.g., using winnowing machine or other machines). The bark/epiderm was separated and fed into organic feed processing plant to be processes into feeds, and the debarked stem was delivered to a cutting machine.

Step (3) Cutting/Slicing: The caragana stem raw materials were cut into segments, each at a length of about 3-4 cm, preferably in oblique cut, in order to increase the area for water penetration.

Step (4) Soaking & Washing: The harvested caragana stem materials were soaked with cold water in a retting pool or tank and washed to get rid of dirt and other debris on its surface. Then, the caragana stem materials were soaked in water at natural temperatures for a time period of about 10-12 hours, or until the stem are totally retted and swelled. After repeat soaking, the solution liquid became turbid. Then, after flocculation and sedimentation, the supernatant clear solution was recycled for re-use. The precipitates and sediments were discharged and abated into a biogas tank for fermentation and producing biogas.

The above four Steps can be performed in separate batches from time to time or step-by-step continuously.

(B) a Fiber-Manufacturing Phase:

from Step 5 (Easing) to Step 12 (Biological pulping solution production).

Step (5) Easing: The soaked and swelled stem raw materials are delivered into a kneading, threading, and/or rolling machine or a roller to be rolled and squeezed and changed into woody hairy structures. The kneading machine to destroy the structure of woody segment and loosen it into timber filaments so that the bacteria culture can easily penetrate inside and degrade the segments.

Step (6) Biodegradation: The treated raw materials were put into a biodegradation bin or pot to be soaked and mixed inside a bacterial culture solution, for example, inside the bacterial culture solution as prepared from Example 1. The weight ratio of the eased raw materials and the bacterial culture solution is 1:6. The temperature was maintained at about 35-40° C. for a time period of about 28-32 hours. Generally, the conditions of biological degradation reaction are adjusted according to the growth conditions of the bacterial culture used so as to increase specific bacterial degradation.

Step (7) Steam Sterilization: Biodegraded materials were removed from the degradation pot, drained, and sterilized to remove bacteria. Then, the biodegraded materials were placed inside a steam pot and steam was used to passing through them for about 10-30 minutes. The biodegraded materials were then delivered from the output of the steam pot into a pulping machine.

Step (8) Coarse Pulping: The sterilized biodegraded materials were transferred to a high concentration grinder for a period of coarse pulping and forming fiber bundles.

Step (9) Fine Pulping: The coarse pulp from the above step were transferred into another high concentration grinder for fine grinding and separating the fiber bundles into individual single fibers.

Step (10) Pulp Screening: The pulp solutions after grinding at least twice may include small portion of fiber bundles. The fiber bundles were repeatedly screened, filtered and re-grinded again to obtain individual single fibers.

Step (11) Pulp Washing: The pulp obtained after coarse pulping and fine pulping were further mechanically grinded. Most of the fibers were curved, twisted, and deformed, and they need to be extracted by soaking in warm water to change, draft and extend any curved fiber structures formed during pulping and grinding, thereby easing the pulping fibers.

Step (12) Paper Board: The above-processed pulping solutions were transferred into a papering machine for manufacturing paperboards in a paper-making process.

(C) A By-Product Generating Phase:

Step A-Step C.

Step A: Organic Feeds. The barks of the prepared Caragana raw materials at Step (3) are rich in nutrients and can be fermented into feeds for cattle and sheep.

Step B: Bio-organic fertilizer. After repeated soaking and swelling at Step (4), the biodegraded material solution became turbid and was delivered to go through flocculation and sedimentation. The resulting supernatant solution was recycled for future use. Then, the precipitates and sediments were discharged and delivered into a biogas pool to be fermented and produce biogas, which can be used to heat a coal and biogas compatible boiler and reduce coal consumption.

Step C: Bio-organic Fertilizer. After fermentation in the biogas pool, the generated biogas residues, slurries, biogas liquids are rich bio-organic fertilizers. So the liquid residue/slurry solutions can be used as fertilizers for agricultural crops and nutrient solutions for flowers. The remaining solid particles and residues can be used as base fertilizers. All such fertilizers are green fertilizers.

The physical properties and index measurements of the paper after the pulping process are shown in Table 4 as below. The data results have shown the reach of Class AA level of excellence for corrugated paper (as seen in Table 6):

TABLE 4

| | Caragana | Eucalyptus | Poplar |
|---|---|---|---|
| Free flowing per ml | 125 | 215 | 290 |
| Weight g/cm² | 115 | 118.5 | 112.9 |
| Whitening % ISO | 40.3 | 37.98 | 48.32 |
| Bulk level cm³/g | 1.82 | 1.85 | 2.27 |
| Tear Index mN m²/g | 7.10 | 3.96 | 3.74 |
| Tensile Index N m/g | 39.03 | 51.3 | 35.8 |
| Burst Index kpa m²/g | 1.62 | 2.2 | 1.5 |
| Ring crush strength index N m/g | 9.6 | 10.2 | 9.3 |

### Example 9

### A Biological Pulping Process Using a Bacterial Culture and Herbal Raw Materials

Using wheat straw as an example of raw materials to specifically illustrate a method of making a biological pulping solution from herbal raw material. As to other herbal raw materials, such as straw, rice straw and cornstalk, a biological pulping process can be carried out according to the process described herein.

When using wheat straw as an example of raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 2:2:1:2:3. When using rice straw as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 1:3:1:2:3. When using cornstalk as raw materials, the weight ratio of a complex microbial flora bacterial culture was configured to be Bacillus:Rheinheimera tangshanensis:Acinetobacter lwoffii:Pseudomonas fluorescens:Wickerhamomyces anomalus at a weight ratio of 2:2:2:1:3.

TABLE 5

| | Wheat Straw | Rice Straw | Cornstalk |
|---|---|---|---|
| Pulp level ° SR | 48 | 46 | 45 |
| Weight g/cm² | 60.2 | 62.5 | 57.1 |
| Whitening % ISO | 19.9 | 23.5 | 20.8 |
| Bulk level cm³/g | 2.08 | 2.43 | 2.80 |
| Tear Index mN m²/g | 3.39 | 4.77 | 2.92 |
| Tensile Index N m/g | 70.9 | 47.8 | 43.9 |
| Burst Index kpa m²/g | 3.09 | 1.97 | 1.82 |

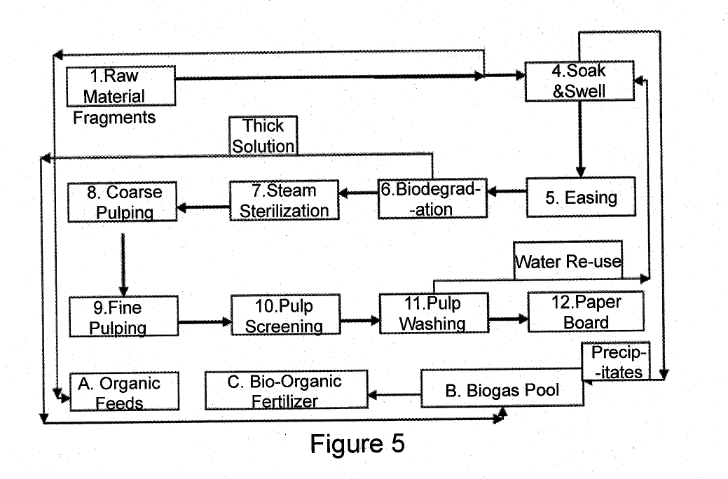

According to the embodiments of FIG. 5, the process of a biological pulping solution system generally includes three phases: a preparation phase, a fiber-manufacturing phase and a by-product generating phase.

TABLE 6
Quality Index of Crude Corrugated Paper

| | | Rule | | | |
|---|---|---|---|---|---|
| | | Level of | Best | Top | Qualified |
| Name of the Index | Unit | Excellence | Product | Product | Product |
| Weight Quantified | g/cm² | AAA | (80, 90, 100, | (80, 90, 100, 110, | |
| (80, 90, 100, 110, | | AA | 110, 120, 140, | 120, 140, 160, 180, | |
| 120, 140, 160, 180, | | A | 160, 180, 200) + 4% | 200) + 5% | |
| 200) | | | | | |
| Tightness (no less | g/cm³ | AAA | 0.55 | 0.5 | 0.45 |
| than) | | AA | 0.53 | | |
| | | A | 0.50 | | |
| Vertical | km | AAA | 5.0 | 3.75 | 2.5 |
| Torn Length (no less | | AA | 4.5 | | |
| than) | | A | 4.3 | | |
| Cross-Sectional | N m/g | AAA | 7.5 | | |
| Pressure Index | | | 8.5 | | |
| cm³/g | | | 10.0 | | |
| | | | 11.5 | | |
| | | AA | 7.5 | 5.0 | 3.0 |
| | | | 7.5 | 5.3 | 3.5 |
| | | | 9.0 | 6.3 | 4.4 |
| | | | 10.5 | 7.7 | 5.5 |
| | | A | 6.5 | | |
| | | | 6.8 | | |
| | | | 7.7 | | |
| | | | 9.2 | | |

(A). A Preparation Phase:

from Step 1 (prepare raw material) to Step 4 (soak & swell. Wheat straw raw materials were cut into segments, each at a length of about 4-5 cm, and then soaked & washed with cold water in a soaking, wetting, retting pool or tank so as to first wash away any dirt and other debris on the surface of the raw materials. The wheat straw raw materials were soaked with water at natural temperature until the wheat straw raw materials were totally retted and swelled, for example, for about 10-12 hours. After repeat soaking, the solution liquid became turbid. Then, after flocculation and sedimentation, the supernatant clear solution was recycled for re-use. The precipitates and sediments were discharged and abated into a biogas tank for fermentation and producing biogas.

(B) A Fiber-Manufacturing Phase:

from Step 5 (Easing) to Step 12 (Additive cellulose production; and (C) A by-product generating phase: Step A-Step C as seen in Example 8. The bacteria culture solution used are prepared the same as described in Example 1. During the biodegradation process, the weight ratio of the treated raw materials and the bacteria culture solution is 1:8. The physical properties and measurements of the obtained fibers are shown in Table 5.

Although the present invention has been disclosed in the preferred embodiment described above, however it is not intended to limit the present invention, any ordinary skill in the art, without departing from the spirit and scope of the present invention, the inner and it is intended that modifications and improvements, Therefore, the scope of the invention as defined in claim depending on whichever.

What is claimed:

1. A complex microbial flora, comprising a first bacteria selected from the group consisting of Pseudomonas fluorescens of deposit number CGMCC No. 5974, and a second bacteria selected from the group consisting of Bacillus. sp. under deposit number CGMCC No. 5971, Rheinheimera tangshanensis under deposit number CGMCC No. 5972, Acinetobacter lwoffii under deposit number CGMCC Deposit No. 5973, and Wickerhamomyces anomalus under deposit number CGMCC No. 5975, and any combinations thereof.

2. The complex microbial flora of claim 1, comprising Pseudomonas fluorescens CGMCC Deposit No. 5974, and Wickerhamomyces anomalus under deposit number CGMCC No. 5975.

3. The complex microbial flora of claim 1, wherein the complex microbial flora is selected from the group consisting of a mixture of Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii:Pseudomonas fluorescens at a weight ratio of 1-2:1-2:1-2; Bacillus:Rheinheimera tangshanensis:Acinetobacter lwoffii:Pseudomonas fluorescens:Wickerhamomyces anomalus at a weight ratio of 3:1:2:2:2; Bacillus sp.:Acinetobacter lwoffii:Pseudomonas fluorescens:Wickerhamomyces anomalus at a weight ratio of 2-3:1-2:1-2:2-3; Bacillus sp.:Acinetobacter lwoffii:Pseudomonas fluorescens at a weight ratio of 2-3:1-2:1-2; Acinetobacter lwoffii:Pseudomonas Fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3; Bacillus sp.:Rheinheimera tangshanensis:Pseudomonas fluorescens:at a weight ratio of 2-3:1-2:1-2; Bacillus sp.:Rheinheimera tangshanensis:Pseudomonas fluorescens:Wickerhamomyces anomalus at a weight ratio of 2-3:1-2:1-2:2-3; Rheinheimera tangshanensis:Pseudomonas fluorescens:Wickerhamomyces anomalus at a weight ratio of 1-2:1-2:2-3; and Bacillus sp.:Pseudomonas fluorescens:Wickerhamomyces anomalus at a weight ratio of 2-3:1-2:2-3.

4. A method for preparing textile fibers, comprising:
preparing a bacterial culture solution using the complex microbial flora of claim 1 in a desired weight ratio of a complex microbial flora solution;
preparing raw materials by preparing and cutting hemp raw materials into fragments and sections, and soaking the raw materials inside a swelling pool to swell the fragments and sections of raw materials;
producing fibers, comprising:
biodegradation, soaking the raw materials with the bacterial culture solution;
steam sterilization by removing and draining the biodegraded materials from the bacteria culture solution, and passing the biodegraded materials through steam sterilization;
obtaining fibers by coarsely grinding the sterilized materials for a period of time to obtain fiber bundles; finely grinding the fiber bundles to disperse the fiber bundles into individual single fibers; screening and filtering the fiber bundles and re-grinding them repeatedly for several times until all of them are grinded into single fibers;
drying and combing the single fibers by soaking the single fibers in warm water, and drying and combing the single fibers for making textile fibers.

5. The method of claim 4, wherein the complex microbial flora is selected from the group consisting of a mixture of: a weight ratio of: Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii:Pseudomonas fluorescens at a weight ratio of 1-2:1-2:1-2; Bacillus:Rheinheimera tangshanensis:Acinetobacter lwoffii:Pseudomonas fluorescens:Wickerhamomyces anomalus at a weight ratio of 3:1:2:2:2; Bacillus sp.:Acinetobacter lwoffii:Pseudomonas fluorescens:Wickerhamomyces anomalus at a weight ratio of 2-3:1-2:1-2:2-3; Bacillus sp.:Acinetobacter lwoffii:Pseudomonas fluorescens at a weight ratio of 2-3:1-2:1-2; Acinetobacter lwoffii:Pseudomonas fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3; Bacillus sp.:Rheinheimera tangshanensis:Pseudomonas fluorescens:at a weight ratio of 2-3:1-2:1-2; Bacillus sp.:Rheinheimera tangshanensis:Pseudomonas fluorescens:Wickerhamomyces anomalus at a weight ratio of 2-3:1-2:1-2:2-3; Rheinheimera tangshanensis:Pseudomonas fluorescens:Wickerhamomyces anomalus at a weight ratio of 1-2:1-2:2-3; and Bacillus sp.:Pseudomonas fluorescens:Wickerhamomyces anomalus at a weight ratio of 2-3:1-2:2-3.

6. The method of claim 4, wherein bacteria density in the bacteria culture solution is above 60,000,000/ml.

7. The method of claim 4, wherein a swelling time for the raw materials is about 10 to 12 hours.

8. The method of claim 4, wherein a biodegradation temperature is maintained at about 35-40° C. for about 32-36 hours.

9. The method of claim 4, wherein the weight ratio of the raw materials and the bacteria culture solution is 1:6-9.

10. A method for preparing celluloses to be used as additives, comprising:
preparing a bacterial culture solution using the complex microbial flora of claim 1 in a desired weight ratio of a complex microbial flora solution;
preparing raw materials by preparing and cutting hemp raw materials into fragments and sections, and soaking the raw materials inside a swelling pool to swell the fragments and sections of raw materials;
producing fibers, comprising:
easing the soaked and swelled raw materials;
biodegradation, soaking the raw materials with the bacterial culture solution;
steam sterilization by removing and draining the biodegraded materials from the bacteria culture solution, and passing the biodegraded materials through steam sterilization;
obtaining fibers by coarsely grinding the sterilized materials for a period of time to obtain fiber bundles; finely grinding the fiber bundles to disperse the fiber bundles into individual single fibers; screening and filtering the fiber bundles and re-grinding them repeatedly for several times until all of them are grinded into single fibers;
soaking the single fibers in warm water; drying and sterilizing the single fibers;
extracting the single fibers into cellulose to be used as additives.

11. The method of claim 10, wherein the complex microbial flora is selected from the group consisting of a mixture of: Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii:Pseudomonas fluorescens at a weight ratio of 1-2:1-2:1-2; Bacillus:Rheinheimera tangshanensis:Acinetobacter lwoffii:Pseudomonas fluorescens:Wickerhamomyces anomalus at a weight ratio of 3:1:2:2:2; Bacillus sp.:Acinetobacter lwoffii:Pseudomonas fluorescens:Wickerhamomyces anomalus at a weight ratio of 2-3:1-2:1-2:2-3; Bacillus sp.:Acinetobacter lwoffii:Pseudomonas fluorescens at a weight ratio of 2-3:1-2:1-2; Acinetobacter lwoffii:Pseudomonas fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3; Bacillus sp.:Rheinheimera tangshanensis:Pseudomonas fluorescens:at a weight ratio of 2-3:1-2:1-2; Bacillus sp.:Rheinheimera tangshanensis:Pseudomonas fluorescens:Wickerhamomyces anomalus at a weight ratio of 2-3:1-2:1-2:2-3; Rheinheimera tangshanensis:Pseudomonas fluorescens:Wickerhamomyces anomalus at a weight ratio of 1-2:1-2:2-3; and Bacillus sp.:Pseudomonas fluorescens:Wickerhamomyces anomalus at a weight ratio of 2-3:1-2:2-3.

12. The method of claim 10, wherein bacteria density in the bacteria culture solution is above 60,000,000/ml.

13. The method of claim 10, wherein the weight ratio of the raw materials and the bacteria culture solution is 1:6-9.

14. A method for preparing biological bacteria solution pulp, comprising:
preparing a bacterial culture solution using the complex microbial flora of claim 1 in a desired weight ratio of a complex microbial flora solution;
preparing raw materials by preparing and cutting hemp raw materials into fragments and sections, and soaking the raw materials inside a swelling pool to swell the fragments and sections of raw materials;
producing a biological pulping solution, comprising:
easing the soaked and swelled raw materials by kneading, threading and rolling;
biodegradation, soaking the raw materials with the bacterial culture solution;
steam sterilization by removing and draining the biodegraded materials from the bacteria culture solution, and passing the biodegraded materials through steam sterilization;
coarsely grinding the sterilized materials for a period of time to obtain fiber bundles;
finely grinding the fiber bundles to disperse the fiber bundles into individual single fibers;
screening and filtering the fiber bundles and re-grinding them repeatedly for several times until all of them are grinded into single fibers;
washing and soaking the biological pulping solution in warm water and using the biological pulping solution for making paper board.

15. The method of claim 14, wherein the complex microbial flora is selected from the group consisting of a mixture of: Bacillus sp.:Rheinheimera tangshanensis:Acinetobacter lwoffii:Pseudomonas fluorescens at a weight ratio of 2-3:1-2:1-2:1-2; Bacillus sp.:Rheinheimera tangshanensis:Acinetobacter lwoffii:Pseudomonas fluorescens:Wickerhamomyces anomalus at a weight ratio of 2-3:1-2:1-2:1-2:2-3; Rheinheimera tangshanensis:Acinetobacter lwoffii:Pseudomonas fluorescens at a weight ratio of 1-2:1-2:1-2; Bacillus:Rheinheimera tangshanensis:Acinetobacter lwoffii:Pseudomonas fluorescens:Wickerhamomyces anomalus at a weight ratio of 3:1:2:2:2; Bacillus sp.:Acinetobacter lwoffii:Pseudomonas fluorescens:Wickerhamomyces anomalus at a weight ratio of 2-3:1-2:1-2:2-3; Bacillus sp.:Acinetobacter lwoffii:Pseudomonas fluorescens at a weight ratio of 2-3:1-2:1-2; Acinetobacter lwoffii:Pseudomonas fluorescens:Wickerhamomyces anomalus at a weight ratio of 1-2:1-2:2-3; and Rheinheimera tangshanensis:Acinetobacter lwoffii:Pseudomonas fluorescens:Wickerhamomyces anomalus at a weight ratio of 1-2:1-2:1-2:2-3; Bacillus sp.:Rheinheimera tangshanensis:Pseudomonas fluorescens:at a weight ratio of 2-3:1-2:1-2; Bacillus sp.:Rheinheimera tangshanensis:Pseudomonas fluorescens:Wickerhamomyces anomalus at a weight ratio of 2-3:1-2:1-2:2-3; Rheinheimera tangshanensis:Pseudomonas fluorescens:Wickerhamomyces anomalus at a weight ratio of 1-2:1-2:2-3; and Bacillus sp.:Pseudomonas fluorescens:Wickerhamomyces anomalus at a weight ratio of 2-3:1-2:2-3.

16. The method of claim 14, wherein bacteria density in the bacteria culture solution is above 60,000,000/ml.

17. The method of claim 14, wherein a swelling time for the raw materials is about 10 to 12 hours.

18. The method of claim 14, wherein a biodegradation temperature is maintained at about 35-40° C. for about 32-36 hours.

19. The method of claim 14, wherein the weight ratio of the raw materials and the bacteria culture solution is 1:6-9.

20. The method of claim 14, wherein after soaking the raw materials, the bacteria culture solution go through flocculation and sedimentation, wherein a supernatant solution is obtained and recycled, precipitates and sediments are discharged and delivered into a biogas pool to be fermented and produce biogas.
